(19) United States
(12) Patent Application Publication
(10) Pub. No.: US 2024/0342426 A1
(43) Pub. Date: Oct. 17, 2024

(54) ANAESTHETIC BREATHING CIRCUIT FOR SMALLER-SIZED MAMMALS

(71) Applicant: Colin DUNLOP, East Ryde NSW (AU)

(72) Inventor: Colin DUNLOP, East Ryde NSW (AU)

(21) Appl. No.: 18/294,578

(22) PCT Filed: Aug. 4, 2022
(86) PCT No.: PCT/AU2022/050840

§ 371 Date: Feb. 2, 2024

(30) Foreign Application Priority Data
Aug. 5, 2021 (AU) 2021104963
Nov. 10, 2021 (AU) 2021903603

(51) Int. Cl. A61M 16/18; A61M 16/04; A61M 16/08; A61M 16/10; A61M 16/20

(52) U.S. Cl. CPC A61M 16/18; A61M 16/0463; A61M 16/0891; A61M 16/1095; A61M 16/208; A61M 2250/00

(57) ABSTRACT

An anaesthesia non-rebreathing circuit for a smaller-sized mammal, the anaesthesia non-rebreathing circuit is a Lack circuit having an adjustable pressure limited valve disposed at the exhalation port located at the fresh gas inlet end of the circuit. The fresh gas delivered by a first conduit from a vaporiser to a patient adaptor. The circuit having a heating arrangement for heating gas within said first conduit.

### TECHNICAL FIELD

This invention relates to an anaesthetic breathing circuit for use with a small mammal. In particular, the present invention is described with reference to a Lack circuit for use with a smaller-sized mammal which weighs less than 5 kgs, such as a cat or small dog during surgery or other procedures. The anaesthetic breathing circuit will also be particularly described with reference to a connector piece for connecting the breathing tubes of that anaesthetic breathing circuit to an endotracheal tube.

### BACKGROUND

Over eighty percent of anaesthetized dogs and cats weigh less than 15 kg, and many weigh less than 5 kg. Like that of human infants/neonates, their small body mass relative to large body surface makes them prone to sever hypothermia (temperature rapidly falling to 32° C.).

There are various types of breathing circuits used in veterinary anaesthesia, including both rebreathing (circle) circuits and non-rebreathing circuits. Rebreathing circuits, which are commonly used on adult human patients of 40 kg and over, are also used in veterinary medicine. One common type of veterinary anaesthesia rebreathing circuit is that disclosed in U.S. Pat. No. 9,717,878 (Dunlop) and marketed as the DARVALL Stingray™ Circle Absorber. In a rebreathing circuit the flow of gas through the machine is circular: reservoir bag-inhalation valve-inspiration hose-animal-expiration hose-exhalation valve-carbon dioxide canister-back to the inhalation valve. Excess gas flow is vented via the waste gas (one-way) valve outlet to the scavenge system. The advantages of the rebreathing circuit are as follows. Firstly, it is economical as expired oxygen and anaesthetic vapor are re-circulated and reused, using less oxygen and anaesthetic agent compared with a non-rebreathing system. Secondly, humidification of inspired gas preserves the heat and moisture of the patient. Thirdly, during the absorption of CO₂ in soda lime, heat is generated, thus assisting to further preserve body heat of the patient. Whilst the abovementioned DARVALL Stingray™ Circle Absorber anaesthesia (rebreathing circuit) can be used on mammals in the 2 kg to 80 kg range, it is not typically used on animals weighing less than 10 kg.

International patent publication no. WO2013/037004 to the present applicant, discloses an apparatus and method for maintaining patient temperature during an anaesthetic procedure. The apparatus and method utilises a heating arrangement on the inspired limb of a rebreathing (circle) circuit, thereby minimising the risk of hypothermia.

Smaller mammals, such as cats and smaller dogs weighing less than 10 kg are typically anaesthetised using non-rebreathing circuits, which is a physically simpler system. In this circuit, oxygen flows through a flow meter and into a vaporizer. At this point, gases exiting the vaporizer go directly to a hose for delivery to the patient with no inhalation one-way valves. Exhaled gases pass through another hose and may enter a reservoir bag, but do not enter a CO₂ absorber. The exhaled gas exits the non-rebreathing circuit via the waste gas outlet and then released into a scavenger system.

Whilst several types of non-rebreathing circuits exist, including but not limited to those known as a Bain circuit and a Magill circuit, they are all modifications of the same basic design. They differ in location of fresh gas inflow, position of reservoir bag and location of exhalation port.

On inspiration, fresh gas is inhaled from both the narrow tubing from the anaesthesia machine (vaporizer) and the corrugated tubing leading away from the endotracheal tube connector. Absence of soda lime means rebreathing of CO₂ must be prevented via high oxygen flows. Inadequate flow rates allow CO₂ to be re-breathed and may create respiratory acidosis. Minimum oxygen flowrates of at least 200-500 ml/kg/minute will prevent significant rebreathing in most mammal patients by flushing out expired gases during the pause between breaths.

One such non-rebreathing circuit is a Lack circuit, which is commonly available in a co-axial tube system, or as a “parallel Lack circuit”. This latter “parallel Lack circuit” is a modification of the classic Magill breathing system, namely fresh gas enters the system not at the patient end (typical of most non-rebreathing systems) but at the end near the reservoir bag (or machine/vaporiser end), with a length of corrugated tubing (the inspiratory limb) in between.

A prior art parallel Lack circuit will be described with reference to FIG. 12. In this parallel Lack circuit 20, an adult Y-piece 21 is shown at the patient end, intended to connect to a endotracheal tube (not shown) of a mammal. Extending from Y-piece 21 are two twenty mm ID parallel corrugated tubes, identified in FIG. 12 as lower fresh gas inspiratory tube 22 and upper reservoir tube 23 which extend to circuit block 24 at the “vaporiser end” of circuit 20. Circuit block 24 is fitted with a reservoir bag 25 and a waste gas outlet 26 and has an inlet 27 through which fresh gas (containing anaesthetic agent) is delivered from a vaporiser (not shown). Circuit block 24 is blanked off between the inlet 27 side and waste outlet 26 so that there is no connection therebetween within block 24. During inspiration, exhalation valve (port) 26 closes and the patient inspires fresh gas from the lower inspiration tube 22. During expiration the patient expires into reservoir tube 23. Towards the end of expiration, reservoir bag 25 fills and positive pressure opens valve 26, allowing expired gas to escape via reservoir tube 23. During the “expiratory pause”, fresh gas washes the expired gas out of reservoir tube 23, filling it with fresh gas.

Firstly, the fresh gas flow must be high enough to “wash out” the expired breath each respiratory cycle to prevent rebreathing of CO₂. For virtually all non-rebreathing systems, the minimum recommended flow is 200 ml/kg/min.

Secondly, as they require very high fresh gas flows, namely up to twenty times more than rebreathing (circle) circuits, so up to twenty times more use (cost) of oxygen and inhalation anaesthetic with up to twenty times more “waste gas” produced with potential to “spill” into the immediate working environment and twenty times more atmospheric pollution. As such, when compared to rebreathing circuits, non-rebreathing circuits pose a significantly greater “occupational hazard and safety” risk. The patient actually uses only 5 ml/kg/min of O₂, which is typically one fifth of the fresh gas flow of efficient low-flow circle breathing anaesthesia systems (e.g. Darvall Stingray™—30 ml/kg/min) and one twentieth of the fresh gas flow of commonly used non-rebreathing (Mapleson) systems (200 to 500 ml/kg/min).

Whilst hypothermia is a problem in all anaesthetised animals, there is a higher risk in smaller mammal patients (under 15 kg) with low body mass relative to a large surface area. The onset of hypothermia is exponential, with a rapid heat loss “phase” developing in the first fifteen minutes following induction of anaesthesia causing a 2° C. to 3° C. drop in body temperature. This is because anaesthetic induction drugs cause peripheral vaso-dilation (so venous pooling in the cold periphery eg hands/feet or paws), resulting in the redistribution (shunting) of blood to the cold periphery. A second, slower and linear phase of heat loss occurring over hours of anaesthesia, is associated with radiation of heat from the skin surface and surgical incisions. So thirdly, the development of hypothermia is more likely with non-rebreathing (Mapleson) systems, because of the high flow (up to twenty times that of rebreathing (circle) systems) of cold, dry gas that requires warming to body temperature and 100% humidification by the time every breath reaches the lungs. This problem of hypothermia is significantly greater in mammals, such as cats and dogs, that weigh less than 5 kg.

A mammal having a mass of about 5 kg using a prior art “paediatric Y piece and endotracheal tube” connected to a Lack circuit will as a result of mechanical dead space (MDS) being between 10-17 ml of that “paediatric Y piece and endotracheal tube”, have an Alveolar Ventilation (Va) of about 25 ml, see the Table at page 15 of this specification. Such a 5 kg mammal would typically have a breath of 50-70 ml. Because of the dead space of a standard or paediatric Y-piece (paediatric=10-17 ml) it is difficult to have mammals smaller than 5 kg on a parallel Lack circuit unless you use very high gas flows, typically a minimum of 500 ml/min (or 200 ml/kg/min, whichever is greater) to ensure the fresh gas washes out the expired gas. With such very high, continuous oxygen and anaesthetic flows, it is difficult to heat the fast moving, turbulent gas stream in the inspired limb of such a circuit. Warming of the inspired gas to achieve a temperature at the Y-piece connection of 43-45° C. is only possible when at least one inspired breath volume sits in the heated inspiratory limb (tubing) during the expiratory pause, so the warming gas is almost stationary for one to three seconds. Therefore, it is not possible to heat the inspired gas of most “continuous flow” non-rebreathing systems where the oxygen and anaesthetic gas are delivered close to the patient connection.

Low flow is possible because firstly, the breathing bag is located close to the fresh gas inlet (in all parallel Lack circuits). Secondly, the adjustable pressure limited (APL) waste gas valve at the end of the expiratory limb is adjusted to keep the bag full, by matching the APL positive end-expiratory pressure (PEEP) to the inspiratory O₂ and anaesthetic flow and the force of expiration of the animal (which will increase as animals get larger, therefore expiring larger breath volumes with more “force”).

Two commercially available parallel Lack circuits, are offered by “Burtons veterinary” of the United Kingdom. The first identified as the “Non-disposable Lack circuit” is indicated to suit veterinary patients greater than 10 kg, whilst the second identified as the “Mini-Lack Anaesthetic Breathing System” is indicated for bodyweights in the range of 1 kg-10 kg. Whilst the promotional material of the “Mini-Lack Anaesthetic Breathing System” indicates that it reduces the gas flowrate to be suitable for smaller mammals, this device still uses a conventional open-close pop-off waste gas valve outlet, and because the flow rate is still too high, it does not address the issue of hypothermia which is at a higher risk for smaller-sized mammals.

Because of the high volume and continuous flow of gases through such prior art parallel Lack circuits, you cannot simply heat the moving gas in the inspired limb of the parallel Lack circuit, as is the case with a rebreathing circuit as disclosed in the earlier mentioned international patent publication no. WO2013/037004. This is because with the high volume and continuous flow of gas, the large volume of space in the inspired limb, and the large dead space of the Y-piece connector and endotracheal tube, it simply is not possible to effectively heat the gas as it rapidly passes through the parallel Lack circuit.

As a result, a major disadvantage of these prior art parallel Lack circuits is the potential for hypothermia in smaller-sized mammals, namely those having a mass of less than 5 kg.

In Western veterinary practice, desexing is commonly carried out on cats and dogs in animal shelters prior to their release to (or adoption by) new owners. Whilst most veterinarians do not recommend desexing cats and dogs until they are about four to five months old, the recent practice of animal shelters has been to desex kittens and pups as young as six to ten weeks old. When kittens and smaller young pups are being desexed these animals may commonly weigh 0.5 to 2 kg. As such the potential for hypothermia during anaesthesia is high.

It should also be noted that “mechanical dead space” is dead space in an apparatus in which the breathing gas must flow in both directions as the user breathes in and out, increasing the necessary respiratory effort to get the same amount of usable air or breathing gas, and risking accumulation of carbon dioxide from shallow breaths. It is in effect an external extension of the physiological (anatomical) dead space. In anaesthetic breathing circuits, it is preferable to minimise the mechanical dead space between a mammal and the breathing tubes of the anaesthetic breathing circuit. The effect of mechanical dead space is of particular concern when anaesthetizing smaller mammals, including humans, which weigh 15 kilograms or less.

There are typical scenarios where small mammals, such as cats, small dogs and human infants undergo procedures requiring anaesthesia, where mechanical dead space is a problem. Standard paediatric Y-piece connectors are known for connecting the breathing tubes of an anaesthetic breathing circuit to an endotracheal tube for use in small mammals. For example, standard paediatric 15 mm (outside diameter) Y-piece connectors 101 are shown in FIGS. 1(a) and (1b) respectively, for a cat 110 and an infant 111 are shown.

In FIG. 1(a) cat 110 is shown on its side, as if positioned for a dental procedure (animals need to be anaesthetised for dentistry, a very common procedure) or for surgery of the head, the eyes, or the cars, with the breathing tubes 102 running towards the anaesthetic equipment (not shown) which would be located toward the rear or side of the surgery table (not shown). The anaesthetist (not shown) would sit next to the anaesthesia machine looking towards the feet and tail of cat 110. The dentist or surgeon (also not shown) would stand or be seated at the head end of the surgery table. All connections are shown to enable the breathing circuit tube direction to facilitate this positioning. To achieve the correct breathing circuit tube direction, the endotracheal tube 103, (typically having a 5 mm inside diameter) and which may typically be made of vinyl, needs to extend further than normal (typically extends 3 cm; in this case an additional 4 cm=total 7 cm protruding from the mouth of cat 110). This additional 4 cm endotracheal tube dead space is undesirable.

The “dead space” is the volume within which the expired gas (from cat 110) that contains CO2, must be rebreathed before “fresh gas” not containing CO2 is breathed in. In addition to adding additional mechanical dead space, the extended and bent vinyl endotracheal tube 103 is liable to having its wall kink, thereby occluding the airway.

In FIG. 1(b), infant 111 is shown on its back as if positioned for abdominal surgery with the breathing tubes 102 running towards the anaesthetic equipment (not shown) which would be at the “head” of the surgery table. The anaesthetist (not shown) would sit next to the anaesthesia machine looking toward the infant's head, the surgeon (also not shown) would stand to one side of the abdomen of infant 111. All connections are shown to enable the breathing circuit tube direction to facilitate this positioning. In a similar fashion to the arrangement of cat 110 in FIG. 1(a), to achieve the correct breathing circuit tube direction, the endotracheal tube 103, (typically having a 5 mm inside diameter) and which may also typically be made of vinyl, needs to extend further than normal (typically extends 3 cm; in this case an additional 4 cm=total 7 cm protruding from the mouth of infant 111). Like that of the example of cat 110 in FIG. 1(a), this additional 4 cm endotracheal tube dead space is undesirable.

To avoid or minimize the risk of kinking the vinyl endotracheal tube 103, you could employ the addition of an elbow 104 between the Y-piece connector 101 and the endotracheal tube 103 as shown for the cat 110 and infant 111 in FIGS. 2(a) and 2(b), respectively. However, whilst the use of elbow 104 reduces the risk of kinking endotracheal tube 103, it is at the expense of adding substantially more mechanical dead space of the additional connector (elbow 104), which is undesirable. This additional mechanical dead space also dilutes the concentration of inspired anaesthetic gas, so the patient, say cat 110, is at risk for inadvertent “wake up” during the procedure.

The certain dead space volumes of the prior art endotracheal tube 103 and ninety-degree (90°) elbow 104 are shown as the grey shaded volumes DS3 and DS4 in FIGS. 3(a) and 3(c) respectively. These areas are referred to in comparison calculation within the description of the present specification.

The present invention provides an anaesthesia non-rebreathing circuit in the form of a Lack circuit for use with smaller-sized mammals that overcomes at least one of the problems associated with the prior art.

The Lack circuit of the present invention can be further improved by using patient adaptor (connector piece) which minimises mechanical dead space.

### SUMMARY OF INVENTION

In a first aspect the present invention consists of an anaesthesia non-rebreathing circuit for a smaller-sized mammal, said anaesthesia non-rebreathing circuit is a Lack circuit having an adjustable pressure limited valve disposed at the waste gas port located at the expiratory end of the circuit, with fresh gas delivered by a first conduit from a vaporiser to a patient adaptor, wherein said circuit having a heating arrangement for heating gas within said first conduit.

Preferably said Lack circuit is a parallel Lack circuit and said first conduit is one of two substantially parallel breathing tubes, and the other of the two breathing tubes is a second conduit disposed between said patient adaptor and the expiratory end of the circuit.

Preferably said heating arrangement comprises a heating element disposed on said first conduit, operably connected to a thermostatic control unit.

Preferably in one arrangement said heating element is a spiral wound element extending along a substantial portion of said first conduit.

Preferably in another arrangement said heating element is a strip element extending along a substantial portion of said first conduit.

Preferably said first conduit is no longer than 1.6 m and has a twelve millimetre inside diameter.

Preferably said adjustable pressure limited valve adapted to have its flow adjusted in gradations between 2 cmH2O positive end-expiratory pressure when fully open and graduated to 25 cmH2O fully closed.

Preferably said smaller-sized mammal has a mass of between 0.5 and 5 kg.

Preferably said patient adaptor is a connector piece that connects said first conduit and said second conduit to an endotracheal tube for use with said smaller-sized mammal, said connector piece having a gallery from which radiate first, second and third tube connectors, said first tube connector disposed between said second and third tube connectors, said first tube connector having a first longitudinal axis and being adapted to connect to said first conduit, said second tube connector having a second longitudinal axis and being adapted to connect to said second conduit, and said third tube connector having a third longitudinal axis and adapted to connect to said endotracheal tube, said third tube connector being disposed at an obtuse angle relative to said first tube connector with said third longitudinal axis intersecting said first longitudinal axis within said gallery, and said second tube connector disposed at an acute angle relative to said first tube connector with said second longitudinal axis intersecting said first longitudinal axis outside of said connector piece.

Preferably said third tube connector of said connector piece being disposed at an angle of about one hundred and ten degrees relative to said first tube connector and said second tube connector disposed at an angle of about thirty-five degrees relative to said first tube connector.

Preferably said third tube connector being disposed at an angle of about one hundred and forty-five degrees relative to said second tube connector.

Preferably said endotracheal tube which connects to said third tube connector has an internal diameter of about 5 mm.

In a second aspect the present invention consists of an anaesthesia non-rebreathing circuit for a smaller-sized mammal having a mass between 0.5 kg and 5 kg, said anaesthesia non-rebreathing circuit is a parallel Lack circuit having an adjustable pressure limited valve disposed at the waste gas port located at the expiratory end of the circuit, with fresh gas delivered by a first conduit from a vaporiser to a patient adaptor, said first conduit is one of two substantially parallel breathing tubes, and the other of the two breathing tubes is a second conduit disposed between said patient adaptor and the expiratory end of the circuit, wherein said circuit having a heating arrangement for heating gas within said first conduit, said heating arrangement comprises a heating element disposed on said first conduit, operably connected to a thermostatic control unit.

Preferably in one arrangement said heating element is a spiral wound element extending along a substantial portion of said first conduit.

Preferably in another arrangement said heating element is a strip element extending along a substantial portion of said first conduit.

Preferably said patient adaptor is a connector piece that connects said first conduit and said second conduit to an endotracheal tube for use with said smaller-sized mammal, said connector piece having a gallery from which radiate first, second and third tube connectors, said first tube connector having a first longitudinal axis and being adapted to connect said first conduit, said second tube connector having a second longitudinal axis and being adapted to connect to said second conduit, and said third tube connector having a third longitudinal axis and adapted to connect to said endotracheal tube, said gallery and said first and third tube connectors in combination provide an elbow-like change in direction within said connector piece, with said third longitudinal axis intersecting said first longitudinal axis within said gallery, and said second longitudinal axis intersecting said first longitudinal axis outside of said connector.

Preferably said elbow-like change in direction allows for orientation of breathing tubes relative to said endotracheal tube without the need of an elbow external of said connector piece, thus minimising mechanical dead space between said endotracheal tube and said breathing tubes.

Preferably said third tube connector being disposed at an obtuse angle relative to said first tube connector with said third longitudinal axis intersecting said first longitudinal axis within said gallery and said second tube connector disposed at an acute angle relative to said first tube connector with said second longitudinal axis intersecting said first longitudinal axis outside of said connector.

Preferably said third tube connector being disposed at an angle of about one hundred and ten degrees relative to said first tube connector and said second tube connector disposed at an angle of about thirty-five degrees relative to said first tube connector.

Preferably said third tube connector being disposed at an angle of about one hundred and forty-five degrees relative to said second tube connector.

### BRIEF DESCRIPTION OF DRAWINGS

FIG. 1(a) is a schematic upper perspective view of a cat positioned on its side, for a veterinary procedure, using a prior art conventional Y-piece connector to connect the endotracheal tube that is placed within the cat's mouth to the breathing tubes of an anaesthetic breathing circuit.

FIG. 1(b) is a schematic upper perspective view of a human infant positioned on its back, for abdominal surgery, using a prior art conventional Y-piece connector to connect the endotracheal tube that is placed within the cat's mouth to the breathing tubes of an anaesthetic breathing circuit.

FIG. 2(a) is a schematic upper perspective view of the cat shown in FIG. 1(a) where a right angle (90 degree) elbow is disposed between the endotracheal tube and the conventional Y-piece connector.

FIG. 2(b) is a schematic upper perspective view of the human infant shown in FIG. 1(b) where a right angle (90 degree) elbow is disposed between the endotracheal tube and the conventional Y-piece connector.

FIGS. 3(a), 3(b) and 3(c) schematic views depicting “dead space” within prior art endotracheal tube, paediatric Y-piece and 90° elbow, respectively.

FIG. 4 is a side view of a first embodiment of a connector piece for connecting the breathing tubes of an anaesthetic breathing circuit to an endotracheal tube for use with a mammal in accordance with the present invention.

FIG. 5 is a plan view of the connector piece shown in FIG. 4.

FIG. 6 is an end view of the connector piece shown in FIG. 4.

FIG. 7 is a perspective view of the connector piece shown in FIG. 4.

FIG. 8 is the side view of FIG. 4, with the orientation angles depicted.

FIG. 9 is a schematic view of the connector of FIG. 4 depicting a volume of certain “dead space” there within.

FIG. 10(a) is a schematic view of a cat positioned on its side, for a veterinary procedure, using the connector piece of FIG. 4 to connect the endotracheal tube that is placed within the cat's mouth to the breathing tubes of an anaesthetic breathing circuit.

FIG. 10(b) is a schematic view of a human infant positioned on its back, for abdominal surgery, using the connector piece of FIG. 4 to connect the endotracheal tube that is placed within the cat's mouth to the breathing tubes of an anaesthetic breathing circuit.

FIG. 11(a) is a schematic view of a prior art conventional Y-piece connector with an End-Tidal CO2 connector and CO2 sensor fitted to the expiratory limb.

FIG. 11(b) is a schematic view of the connector of FIG. 4 with an End-Tidal CO2 connector and CO2 sensor fitted to the expiratory limb.

FIG. 12 is a schematic of a prior art parallel Lack circuit, utilising a conventional “pop-off” exhalation valve and a conventional Y-piece.

FIG. 13 is a schematic of a first embodiment of parallel Lack circuit for a smaller-sized mammal utilising the connector piece of FIG. 4

FIGS. 14a, 14b and 14c show the “End-inspiration” phase, “Early expiration” phase and End-expiration” phase of the parallel lack circuit shown in FIG. 13.

FIG. 15 is the key of shaded areas “Fresh Gas”, “Dead Space Gas” and “Alveolar Gas” shown in FIGS. 14a, 14b and 14c.

### BEST MODE OF CARRYING OUT THE INVENTION

In the present specification references to “small mammal”, is a mammal of no more than 15 kg mass. A “small mammal” may include but is not limited to any one of cats, dogs and human infants. Also, in the present specification references to a “smaller-sized mammal”, is a mammal of no more than 5 kg mass. A “smaller-sized mammal” may include but is not limited to cats and dogs.

In the present specification the term “mechanical dead space” (MDS) is used with reference to a mammal patient, such as a small mammal being anaesthetized. It is a volume within an endotracheal tube that is placed within the mouth of a small mammal and any connector piece which connects it to breathing tubes of an anaesthetic breathing circuit. This dead space is the volume within which the expired gas from the small mammal that contains CO2, must be rebreathed before “fresh gas” not containing CO2 is breathed in.

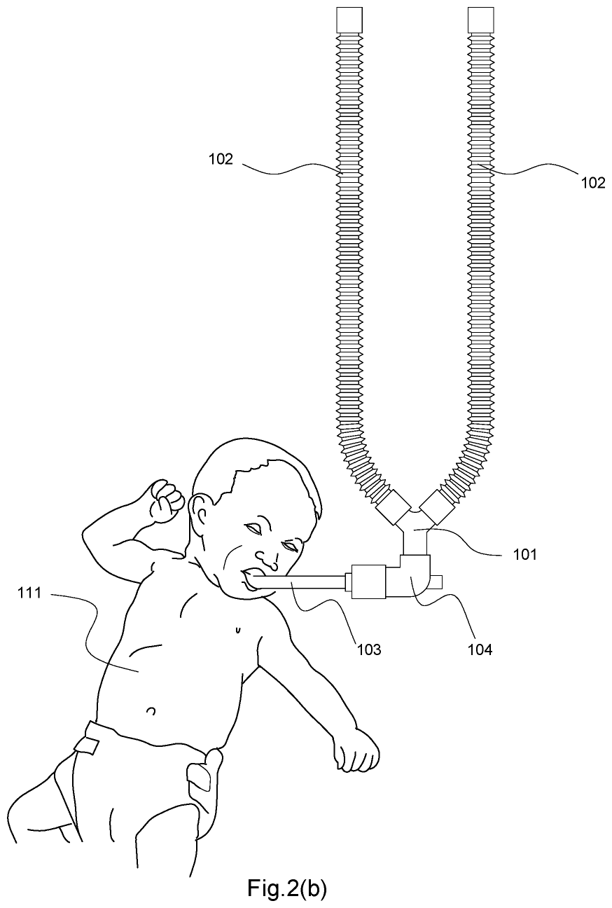

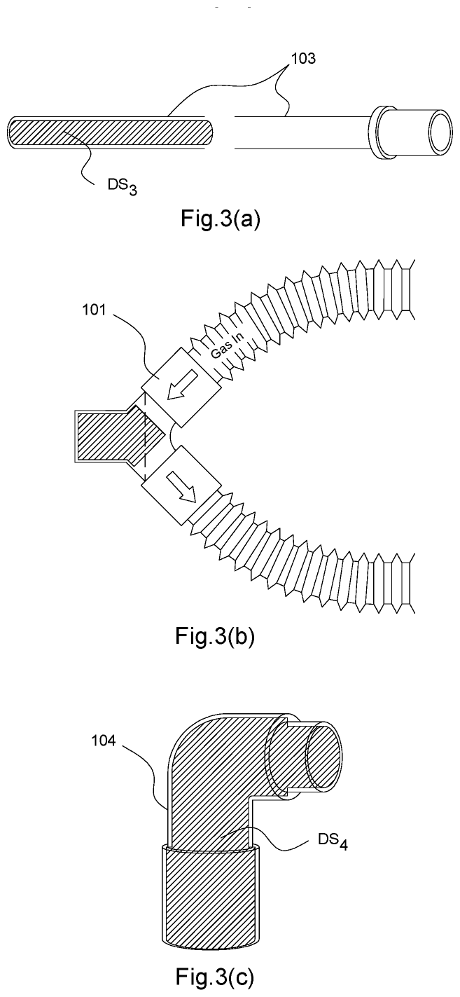

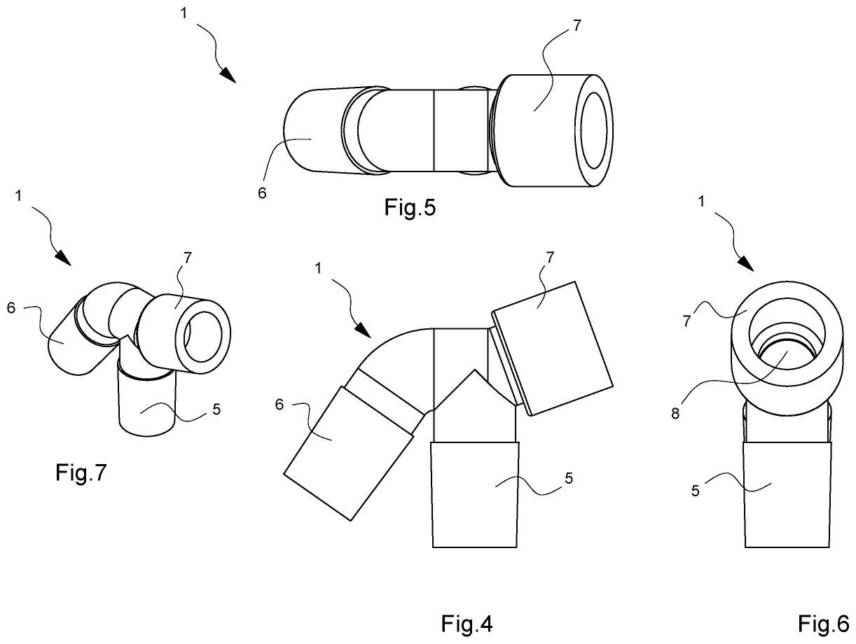

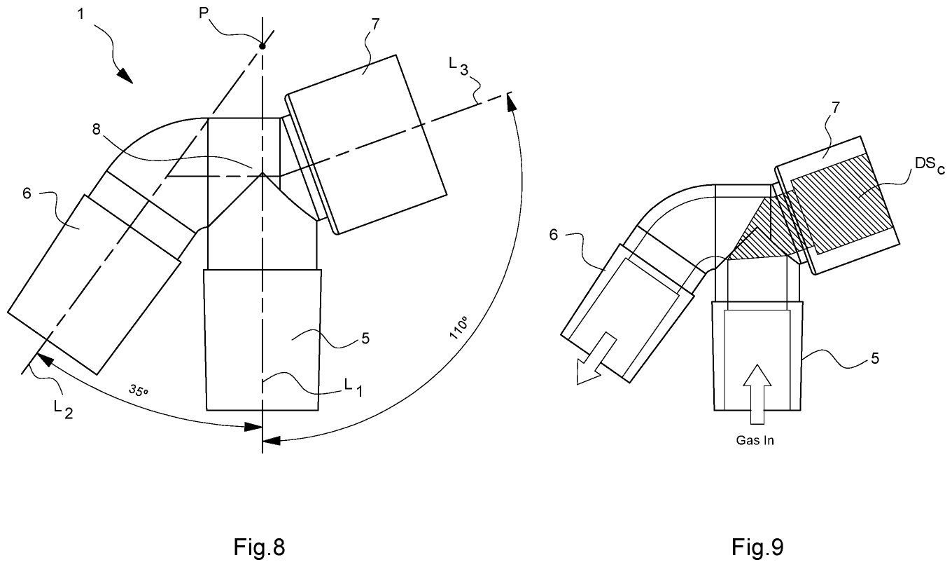

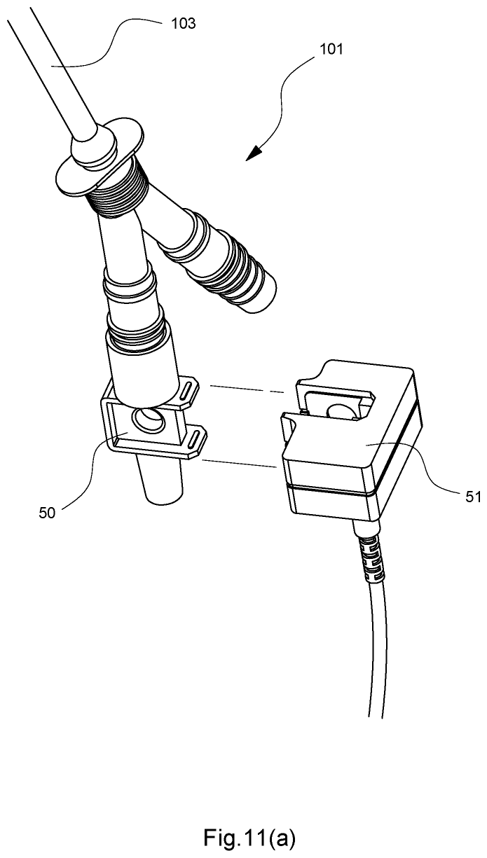

FIGS. 4 to 10(b) depict a connector piece which can be used with the first embodiment of the present invention. Connector piece 1 is intended for procedures and surgery which require a small mammal, such as a cat 10 shown in FIG. 10(a), or human infant shown in FIG. 10(b) to be anaesthetized.

Connector piece 1, which is internally hollow, is for connecting breathing tubes 2 of anaesthetic equipment (not shown) that deliver anaesthetic in a breathable form (anaesthetic gas), to an endotracheal tube 3 that is placed within cat 10 via its mouth as shown FIG. 10(b).

In this embodiment connector piece 1 is preferably made of a plastic material, such as polycarbonate or polypropylene.

In this embodiment, connector piece 1 has three tube connectors (limbs), namely first connector 5, second connector 6 and third connector 7, all of which are tubular in form, and preferably have an outside diameter of 15 mm.

Endotracheal tube 3 is made of vinyl and has an internal diameter of 5 mm.

Disposed internally of connector piece 1, is a gallery 8 about which tube connectors 5, 6 and 7 radiate and interconnect. Connectors 5 and 6 are for connection to connector tubes (hoses) 2, whilst connector 7 is connected to endotracheal tube 3.

Connector 5 has a first longitudinal axis L1 and is adapted to receive and connect to a first of the two breathing tubes 2. Connector 6 has a second longitudinal axis L2 and is adapted to receive and connect to the other (second) of the two breathing tubes 2. Connector 7 having a third longitudinal axis L3 is adapted to receive and connect to endotracheal tube 3.

In this embodiment, longitudinal axis L3 of third connector 7 is disposed at an obtuse angle, namely one hundred-and-ten degrees, relative to first connector 5, with third longitudinal axis L3 intersecting first longitudinal axis L1 of first connector 5 within gallery 8. Second connector 6 is disposed at an acute angle, namely thirty-five degrees, relative to said first connector 5, with second longitudinal axis L2 intersecting first longitudinal axis L1 outside of connector piece 1 at point P. The angle between second connector 6 and third connector 7, namely the sum of one hundred-and-ten degrees and thirty-five degrees, is one hundred-and-forty-five degrees.

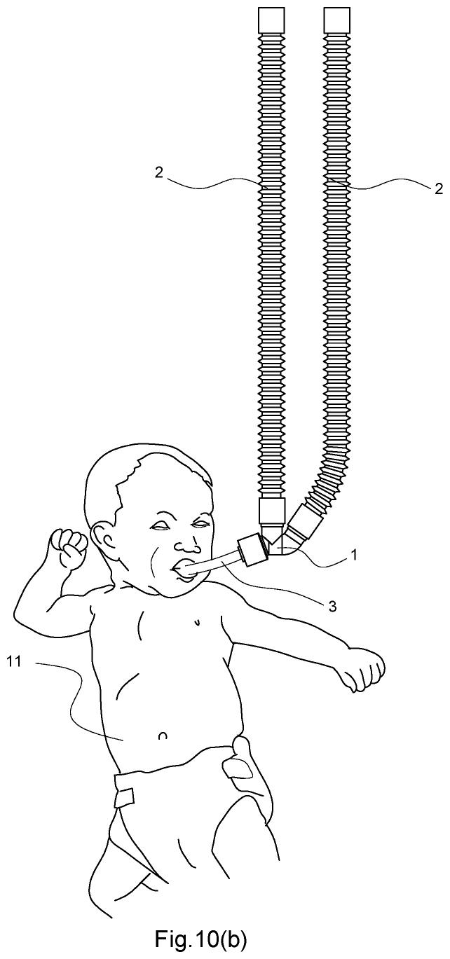

Connector piece 1 has an area of MDS shown as the shaded area DSC in FIG. 9.

Gallery 8 and first and third tube connectors 5,7 in combination, and due to the orientation of longitudinal axes L1 and L3, provide an “elbow-like” change in direction within connector piece 1.

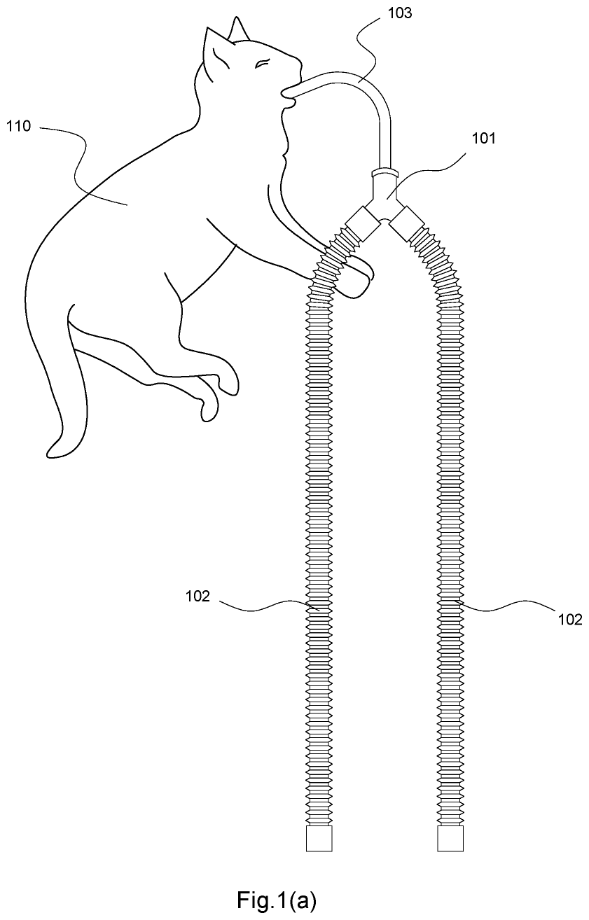

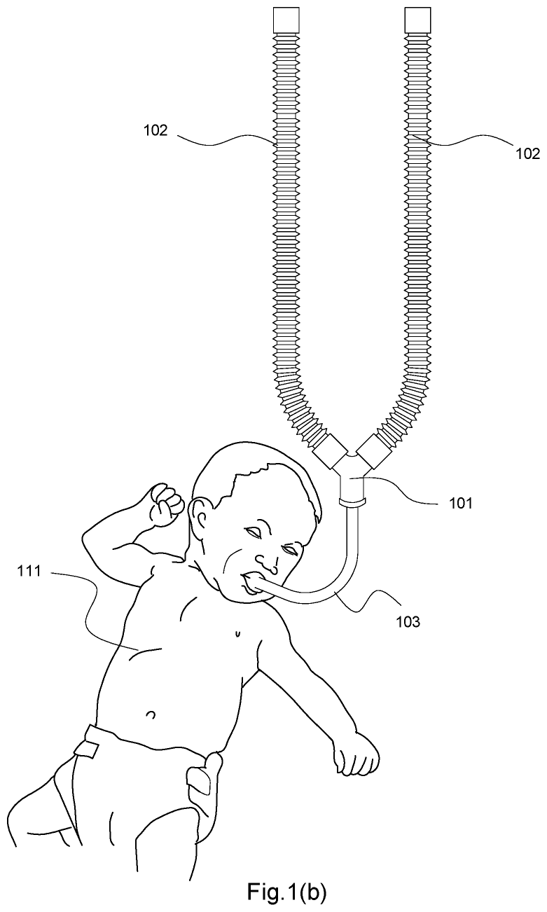

As shown in FIG. 10(a), connector piece 1 of the present invention, with this abovementioned “elbow-like” change in direction within connector 1, allows for similar orientation of breathing tubes 2 relative to the cat 10, as the prior art arrangement, but has eliminated the need for the extra length of endotracheal tube, as required in the prior art shown in FIG. 1(a). Furthermore, the arrangement in FIG. 10(a) has by the orientation to each other of connectors 5, 6 and 7 of connector piece 1, allowed for better directional placement of breathing tubes 2 without the need of an additional elbow 104, as was required in the prior art depicted FIG. 2(a). As, such connector piece 1 with its “elbow-like” change in direction eliminates both the kinking problem of the prior art depicted in FIG. 1(a) and minimises the MDS of both the prior art arrangements of FIG. 1(a) and FIG. 2(a).

As shown in FIG. 10(b), connector piece 1 of the present invention allows for similar orientation of breathing tubes 2 relative to the human infant 11. In this embodiment, like that of cat 10 in FIG. 10(a), connector piece 1 with its “elbow-like” change in direction eliminates both the kinking problem of the prior art depicted in FIG. 1(b) and minimises the MDS of both the prior art arrangements of FIG. 1(b) and FIG. 2(b).

To highlight the minimization of MDS we make the following comparison of the present embodiment to that of the prior art in the following MDS calculations:

### Prior Art Y Piece Connector Arrangement FIGS. 1(a) & 1(b)

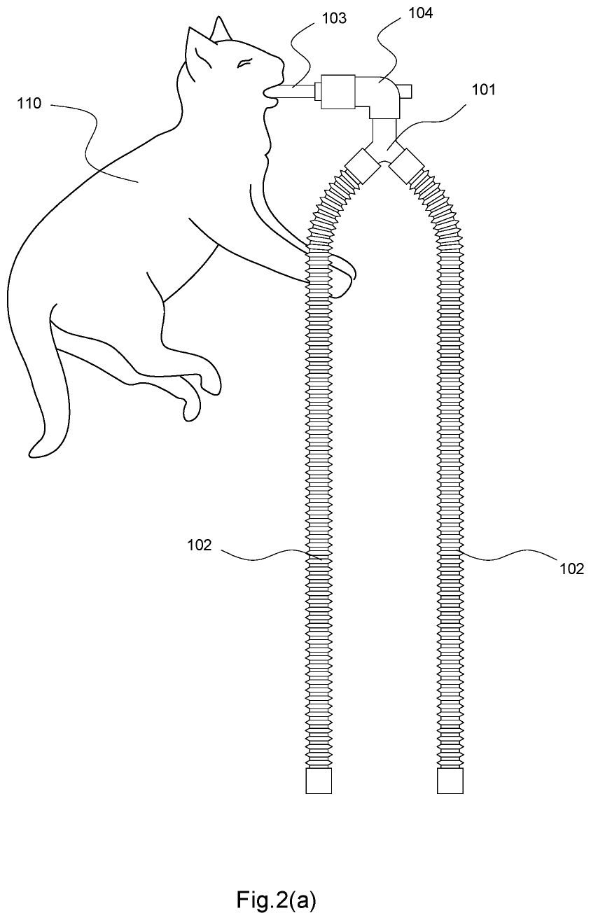

Endotracheal Tube (ET)—5 mm ID
(typical for large 4 kg cat FIG. 1(a) and Human Infant FIG. 1(b))

| | |
|---|---|
| 3 cm projection from mouth (“standard” projection) | 0.6 ml |
| 4 cm projection from mouth to provide | 0.8 ml |
| “bend” in tube (DS₃ in FIG. 3(a)) | |
| MDS total for 7 cm projection from mouth | 1.4 ml |

Standard Paediatric Y-Piece—15 mm OD
Total Volume=14 ml (mechanical dead space is a portion thereof—see FIG. 3(b))
The breathing hose channels: π×0.6252×2.8=3.44 ml
(each side)×2=6.88 ml
Standard Paediatric Y-piece MDS=14−6.88=7.12 ml
Standard Paediatric Y-Piece+7 cm Endotracheal tube projection has a MDS=7.12 ml+9.8 ml=8.52 ml

### Prior Art Y Piece Connector With Elbow Arrangement FIGS. 2(a) & 2(b)

90° elbow adaptor (AAS part No. 8351) dead space shown as DS₄ in FIG. 3(c)=9.8 ml
Standard Paediatric Y-Piece+90° Elbow+3 cm ET Tube projection has an MDS=7.12 ml+9.8 ml+0.6 ml=17.52 ml

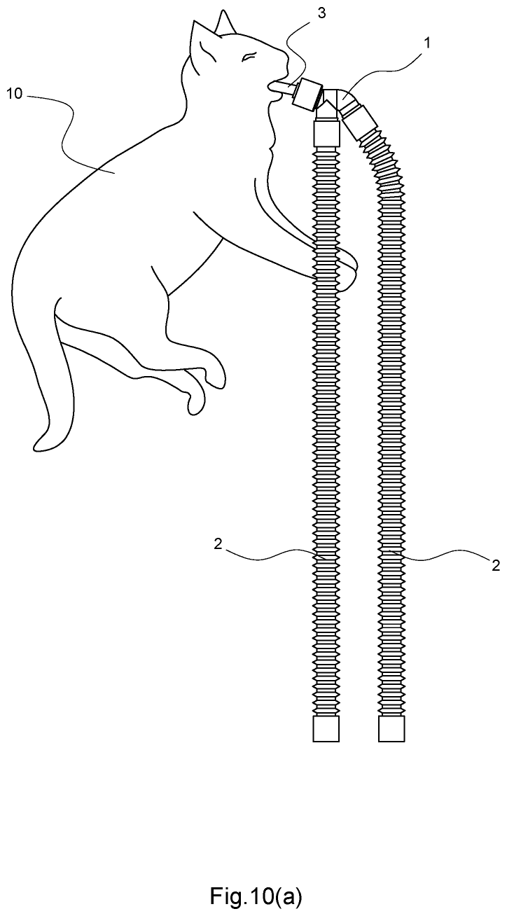

### Present Embodiment Connector Piece 1 Arrangement FIGS. 8(a) and 8(b)

Connector piece 1 (grey shaded area dead space DS_{C})=3.6 ml+the 3 cm ET Tube projection has an MDS=3.6 ml+0.6 ml=4.2 ml

This means that the MDS of the present embodiment is minimized to about 50% when compared to the Y-connector arrangement of FIG. 1(a) and FIG. 1(b), and to about 24% when compared to the elbow arrangement.

To better understand the improvement of the abovementioned preferred embodiment of present invention as described with reference to FIGS. 4 to 10(b), it is important to consider the alveolar ventilation of the mammal.

Alveolar ventilation is the exchange of gas between the alveoli and the external

environment. It is the process by which oxygen is brought into the lungs from the atmosphere and by which the carbon dioxide carried into the lungs in the mixed venous blood is expelled from the body.

### For Cat of Mass 3 kg

Normal Breath 10-15 ml/kg=30-45 ml
Anatomic dead space (trachea etc) is about a third (say 33%)=about 10-15 ml
So net alveolar ventilation=20-30 ml

### For Human Infant of Mass 5 kg

Normal Breath 10-15 ml/kg=50-75 ml
Anatomic dead space (trachea etc)=33%=about 15-25 ml
So net alveolar ventilation=35-50 ml

Any anaesthetic connection dead space reduces alveolar ventilation. If severe, the mammal's blood CO2 rises, anaesthetic level gets lighter (rebreathing dilutes inspired gas concentration), and the mammal may become hypoxic.

Anaesthetic MDS becomes less important once animals/infants (mammals) get over 10 to 15 kg, as they do not require a paediatric Y-piece of the earlier mentioned prior art. For anaesthetic MDS in a connector less than 5% is irrelevant and 10% is okay. However, MDS greater than that is problematic.

We now provide a summary of MDS calculations used for various sizes of small mammal.

### Prior Art Paediatric Y Piece (FIGS. 1(a) & 1(b))

=7.12 ml+4 cm extended ET tube: 0.8 ml=7.92 ml

### Prior Art Paediatric Y Piece+Elbow (FIGS. 2(a) & 2(b))

=7.12 ml+Elbow 9.8 ml=16.92 ml

### Present Embodiment Connector Piece 1 FIGS. 8(a) and 8(b)=3.6 ml

Animal Pulmonary Ventilation: Tidal Volume (Vt)=10 ml/kg−⅓ Anatomic Dead Space (DSa=nose, airway)+Machine DS (DSm)=net Alveolar Ventilation (V_{A})
So calculating this V_{A} for various mammals of 3 kg, 5 kg, 10 kg and 15 kg mass is as follows:
3 kg×10 ml=30 ml Vt−10 ml DSa=20 ml V_{A}
5 kg×10 ml=50 ml Vt−16.7 ml DSa=33.3 ml V_{A}
10 kg×10 ml=100 ml Vt−33 ml DSa=67 ml V_{A}
15 kg×10 ml=150 ml Vt−50 ml DSa=100 ml V_{A}

TABLE
Decrease of net Alveolar Ventilation (VA) for size of mammal

| | 3 kg mammal | 5 kg mammal | 10 kg mammal | 15 kg mammal |
|---|---|---|---|---|
| Connector | (20 ml V_{A}) | (33.3 ml V_{A}) | (67 ml V_{A}) | (100 ml V_{A}) |
| Prior art | 20-7.92 = | 33-7.92 = | 67-7.92 = | 100-7.92 |
| Paediatric Y + | 12.08 ml | 25.08 ml | 59.08 ml | 90 ml |
| extend ET tube | V_{A} = 40% | V_{A} = 24% | V_{A} = 12.6% | V_{A} = 8% |
| 10 ml MDS | decrease | decrease | decrease | decrease |
| Prior art | 20-16.92 = | 33-16.92 = | 67-16.92 = | 100-16.92 = |
| Paediatric Y + | 3.1 ml | 16.1 ml | 50.1 ml | 83.1 ml |
| 90° elbow | V_{A} = 85% | V_{A} = 51% | V_{A} = 25% | V_{A} = 17% |
| 10 ml MDS | decrease | decrease | decrease | decrease |
| embodiment | 20-3.6 = | 33-3.6 = | 67-3.6 = | 100-3.6 |
| Connector | 16.4 ml | 29.4 ml | 63.4 ml | 96.4 ml |
| Piece 1 | V_{A} = 18% | V_{A} = 11% | V_{A} = 5.4% | V_{A} = 3.6% |
| 3.6 ml MDS | decrease | decrease | decrease | decrease |

So as can be seen from this abovementioned table, connector piece 1 significantly minimizes the decrease of Alveolar Ventilation (VA) when compared to the prior art, and this minimization is more significant in the smaller mass (sized) mammals.

In the above referenced embodiment, the small mammals referred to are cats and human infants, however the invention of the present embodiment could be on used on any small mammals including but not limited to dogs, rabbits, macropods, monkeys, and chimpanzees.

It should be understood, that the smaller the mammal, then the size of mechanical dead space has a greater impact, so the real advantages of minimizing MDS by use of connector piece 1, is even greater the smaller the mass of the mammal. This means it is particularly advantageous for use with anaesthetic breathing circuits for domesticated cats whose typical adult mass is 3.5-4.5 kg, and smaller breed dogs, such as Chihuahuas and Pomeranians for example.

Referring to FIG. 4, what should be understood is that whilst connector piece 1 is shown with third connector 7 disposed at an obtuse angle, namely one hundred-and-ten degrees, relative to first connector 5, and second connector 6 is disposed at an acute angle, namely thirty-five degrees, relative to said first connector 5, these angles could vary. When they vary, it is preferable that the angle between second connector 6 and third connector 7 remain at about one hundred-and-forty-five degrees. So, in another not shown embodiment if the obtuse angle between third connector 7 and first connector 5 is increased by five degrees to that shown, namely to one hundred-and-fifteen degrees, then the acute angle between second connector 6 and first connector 5 should preferably be decreased by about five degrees to thirty degrees, thereby maintaining the sum of those two angles between second connector 6 and third connector 7 at about one hundred-and-forty-five degrees. By doing so the orientation of the connectors 5,6,7 maintain a preferable “elbow-like” change in direction within connector piece 1. Likewise, in a further not shown embodiment, if the obtuse angle between third connector 7 and first connector 5 is decreased by five degrees to that shown, namely to one hundred-and-five degrees, then the acute angle between second connector 6 and first connector 5 should preferably be increased by about five degrees to forty degrees, thereby maintaining the sum of those two angles between second connector 6 and third connector 7 at about one hundred-and-forty-five degrees. Likewise, by doing so the orientation of the connectors 5,6,7 maintain a preferable “elbow-like” change in direction within connector piece 1.

The abovementioned embodiment of present invention is also advantageous when used with End-Tidal CO2 (ETCO2) adaptors for smaller mammals, particularly the mid-sized smaller animals from 5-10 kg.

To best show this advantage, it is best to understand the prior art. Any anaesthetic connection dead space reduces alveolar ventilation. In smaller animals/infants this problem is important because rebreathing CO2 causes the animals blood CO2 to rise which increases circulating catecholamines, heart rate, blood pressure and cardiac output. Rebreathing also dilutes the inspired concentration of anaesthetic so anaesthetic level gets lighter and can lead to hypoxia.

The commonest way to continuously measure the CO2 level is by end-expired CO2 sampling (ETCO2=equivalent to alveolar gas sampling) using infrared (IR) gas analysis (CO2 gas absorbs infra-red light in a concentration-dependent manner). Whilst there are two ways of doing this, namely, side-stream sampling or main-stream sampling, the latter is more accurate with smaller mammals.

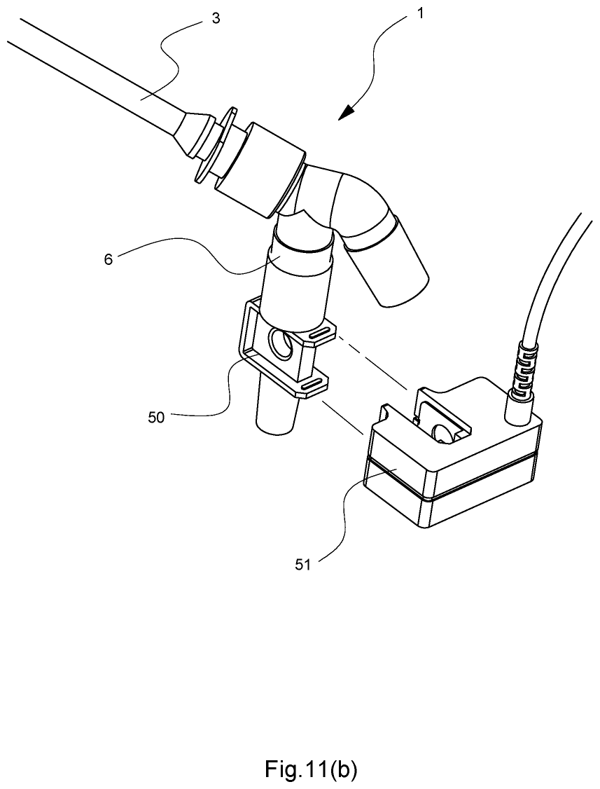

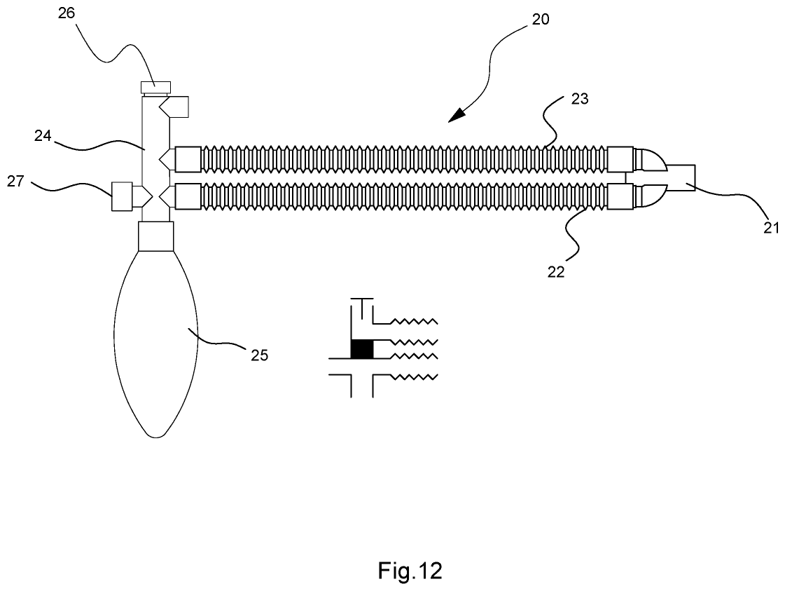

Prior art main-stream sampling for smaller mammals of 5-10 kg mass as shown FIG. 11(a), requires adding a sample ETCO2 connector 50 between endotracheal tube 103 and Y-Piece connectors 101 (earlier shown in FIGS. 1(a) and 1(b)), that the animal/human breaths through. Connector 50 is attached to the expiratory limb of Y-piece connector 101. Connector 50 has a side window. A CO2 sensor 51 incorporating an IR spectrophotometer is attached to connector 50 and measures the CO2 level in real time across the window as the animal breathes in and out. This system is more accurate in small animals and humans where the breath size is small and is preferable over that of the main-stream sampling system, however it does have the potential of adding MDS.

Connector 50 and sensor 51, can be used with connector piece 1, connected to endotracheal tube 3 of the earlier describe embodiment, as shown in FIG. 11(b). Connector 50 is attached to the expiratory limb (second connector 6). In this arrangement, because the MDS has been minimized by use of connector piece 1, the placement of connector 50 and sensor 51 at the expiratory limb (second connector 6), allows for a more accurate CO2 sample for the smallest patient for which this set up is used, namely for small mammals of mass 5 kg to 10 kg.

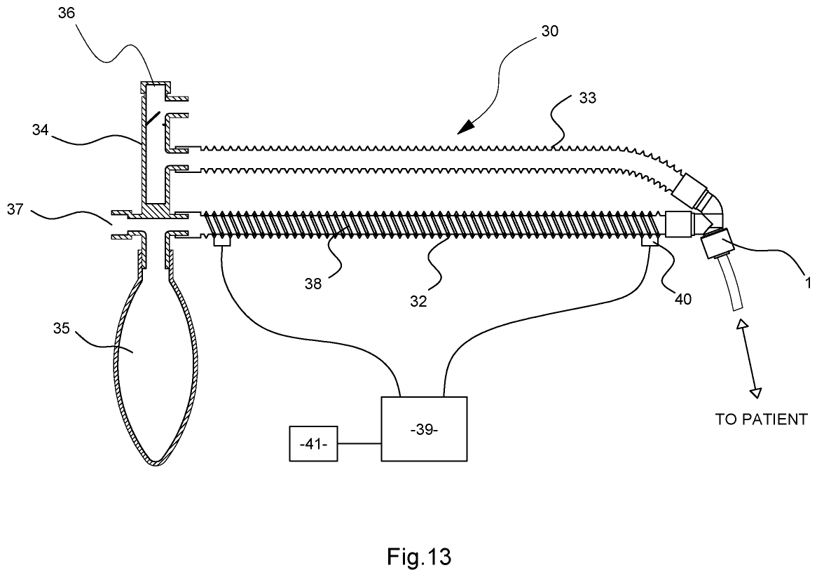

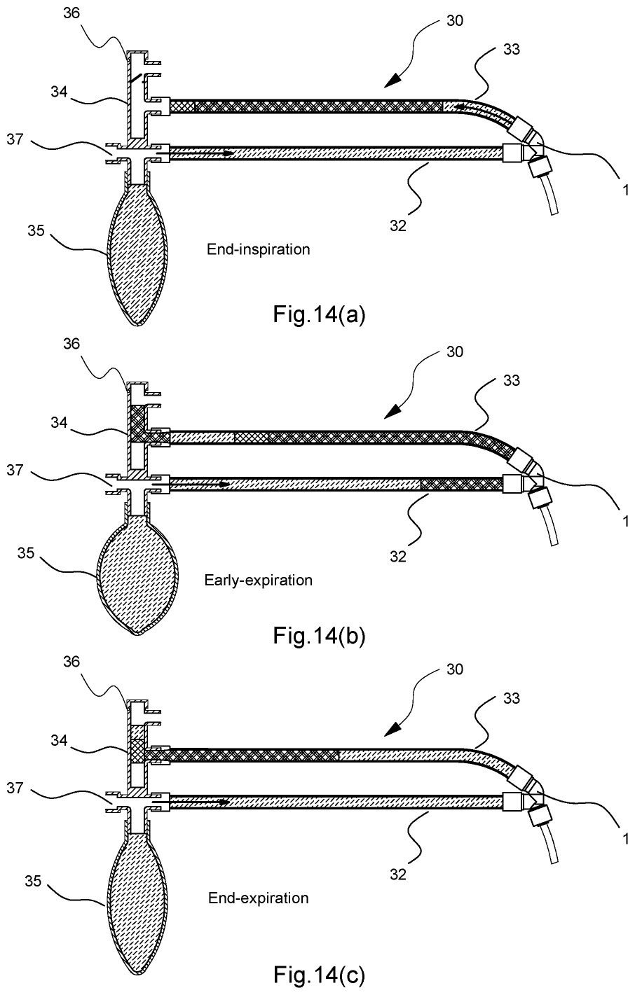

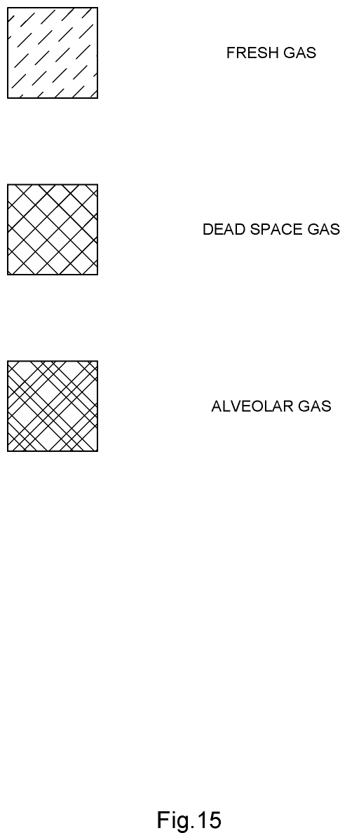

FIGS. 13 and 14a, 14b and 14c depict a parallel Lack circuit 30. Connector piece 1 is shown at the patient end, intended to connect to an endotracheal tube (not shown) of a mammal. Extending from connector 1 are two parallel tubes, identified as fresh gas inspiratory tube (conduit) 32 and reservoir tube (conduit) 33 which extend to circuit block 34 at the “vaporiser end” of circuit 30. Circuit block 34 is fitted with a reservoir bag 35 and exhalation valve 36 at the waste gas port and has an inlet 37 through which fresh gas (containing anaesthetic agent) is delivered from a vaporiser (not shown). Circuit block 34 is blanked off between the inlet 37 side and exhalation valve 36 so that there is no connection therebetween within block 34.

Exhalation valve 36 is a “fail-safe” adjustable pressure-limiting (APL) valve that allows for efficient low gas flows. This APL valve 36 is capable having the flow adjusted in gradations. An APL valve 36 that is suitable has 2 cmH2O positive end-expiratory pressure (PEEP) when fully open and graduated to 25 cmH2O fully closed. From 2 cmH2O to 25 cmH2O is a graduated scale with linear increase in PEEP as it progresses from full open to full closed.

One such APL Valve 36 which is suitable is the “Paediatric APL Valve” by Intersurgical™.

When this Paediatric APL Valve by Intersurgical™ is fully open:
resistance to flow=0.4 cm H₂O at 3 L/min continuous flow; and
   it is actuated by expiratory force/pressure of less than 1 cm H₂O (force required to overcome static opening resistance)
   Valve fully closed:
resistance to flow=27 cmH₂O at 4 L/min (30-31 cm H₂O at 10 L/min)−4 L/min/200 ml/kg/min=20 Kg mammal

This Paediatric APL Valve by Intersurgical™ has linear performance from open to closed, so increasing PEEP from 0.4 cm H2O to 27 cm H2O

Fresh gas inspiratory tube 32 preferably has a twelve millimetre (12 mm) inside diameter, and preferably is no longer than 1.6 m. Inspiratory tube 32 is provided with a “heating arrangement”, comprising a heating element 38 and a thermostatic control unit 39 powered by a DC supply 41. Thermostatic control unit 39 controls the heat delivered to heating element 38. In FIG. 13 heating element 38 is a spiral wound element extending along a substantial portion of inspiratory tube 32. However, in an alternative arrangement heating element 38 could be a strip element extending along a substantial portion of inspiratory tube 32.

You could use sixteen millimeter (16 mm) inside diameter tube for the fresh gas inspiratory tube 32. However, it is possible to warm the inspired gas more effectively with 12 mm ID tube 32, compared to 16 mm at the same O2 flows. The 12 mm ID tube is preferred for the small-sized mammals as the fresh gas is “closer” to heater element 3,8 so better and more rapid warming is achieved. A 12 mm tube which is 1.6 m long holds 180 ml gas, which is enough for a 12-18 kg mammal.

One suitable inspiratory tube 32 in combination with the “heating arrangement” is the twelve millimetre (12 mm) inside diameter heated tube (which includes heating element 38) of the DARVALL™ Warm Air Starter Kit, the control unit 39 being the DARVALL™ WARM AIR INSPIRED control unit.

This thermostatic control unit 39, also includes a temperature sensor 40 to sense the temperature of the heated fresh gas passing through inspiratory tube 32, and a temperature display (not shown).

The operation of parallel Lack circuit 30, and its suitability for smaller-sized mammals will now be described with reference to FIGS. 14a, 14b and 14c, which depict the location of Fresh Gas, Dead Space Gas and Alveolar Gas during the respective “End-inspiration”, “Early expiration” and End-expiration” phases.

Dead Space Gas is primarily the gas made up of oxygen (O2) and carbon dioxide (CO2) gasses that are not exchanged across the alveolar membrane in the respiratory tract. This Dead Space gas is from both MDS and the patient's anatomic dead space.

Alveolar Gas is the gas expired by the patient which contains CO2.
APL valve 36 provides a gradually increasing resistance as it is opened or closed to apply some PEEP, typically 2 to 4 cm H₂0, to replace the simple open-close “pop-off” waste gas outlet valve of the prior art. Where the fresh gas flow through inspiratory tube 32 balances the PEEP on APL valve 36, reservoir bag 35 stays “full” at lower fresh gas flows, see FIGS. 14b and 14c, preventing rebreathing of CO₂ but also reducing the fresh gas flow to a minimum flow of about 200 ml/min (sufficient for a 1 kg to 2 kg mammal).
The use of a 1.6 m length of 12 mm ID heated smooth wall tube for inspiration tube 32 with a volume of about 180 ml, means that effectively more than one breath is “stored” in the inspiratory limb, for animals up to 12 kg-18 kg (10-15 ml/kg/breath) permitting effective warming to 45° C. at the connector piece (patient) hose end, at fresh gas flows of 200 ml/min to 2000 ml/min. The temperature of the fresh gas will effectively drop to about 35° C. by the time it is inspired by the patient.
By using connector piece 1, which has an MDS of 3.6 ml, this parallel Lack circuit 30 can be used on mammals with a breath size as small as 7 ml, and this connector piece 1, only increases the dead space from 30% (normal) to 50% (acceptable) so permitting safe, low flow use of parallel Lack circuit 30 on mammals from 0.5 to 2 kg and enable effective warming of the inspired gas to 45° C. at the patient connection.

Trials using this parallel Lack circuit 30 incorporating connector piece 1 have shown that for a smaller-sized mammal of 1 kg and with a “Fresh Gas” flow of 200 ml/min from a Darvall Vaporiser (as disclosed in International Patent Publication No. WO2020/146919) that no rebreathing of CO2 (Alveolar Gas) occurred. Furthermore, this circuit 30 was tested as low as 100 ml/min without rebreathing of CO2. This occurs because of the low inlet gas flow is balanced to the outlet gas flow by APL valve 36 in combination with the minimised dead space provided by connector piece 1. Furthermore because of the lower flow rate, the volume of fresh gas is “almost stationary” in the inspired limb of circuit 30, with each breath is as high as 180 mls, this fresh gas can be heated sufficiently as it passes through tube 32, so that the smaller-sized mammal of 1 kg mass receives warm air heated to 45° C. thereby minimising the risk of hypothermia.

What should be understood is that in this embodiment of parallel Lack circuit 30, connector piece 1 is the preferred “patient adaptor”, as it contributes to minimising mechanical dead space. However, parallel Lack circuit 30 could be used with a conventional Y-piece connector as the “patient adaptor”.

The terms “comprising” and “including” (and their grammatical variations) as used herein are used in an inclusive sense and not in the exclusive sense of “consisting only of”.

1. An anaesthesia non-rebreathing circuit for a smaller-sized mammal, said anaesthesia non-rebreathing circuit is a Lack circuit having an adjustable pressure limited valve disposed at the waste gas port located at the expiratory end of the circuit, with fresh gas delivered by a first conduit from a vaporiser to a patient adaptor, wherein said circuit having a heating arrangement for heating gas within said first conduit.

2. An anaesthesia non-rebreathing circuit as claimed in claim 1, wherein said Lack circuit is a parallel Lack circuit and said first conduit is one of two substantially parallel breathing tubes, and the other of the two breathing tubes is a second conduit disposed between said patient adaptor and the expiratory end of the circuit.

3. An anaesthesia non-rebreathing circuit as claimed in claim 2, wherein said heating arrangement comprises a heating element disposed on said first conduit, operably connected to a thermostatic control unit.

4. An anaesthesia non-rebreathing circuit as claimed in claim 3, wherein said heating element is a spiral wound element extending along a substantial portion of said first conduit.

5. An anaesthesia non-rebreathing circuit as claimed in claim 3, wherein said heating element is a strip element extending along a substantial portion of said first conduit.

6. An anaesthesia non-rebreathing circuit as claimed in claim 1, wherein said first conduit is no longer than 1.6 m and has a twelve millimetre inside diameter.

7. An anaesthesia non-rebreathing circuit as claimed in claim 1, wherein said adjustable pressure limited valve adapted to have its flow adjusted in gradations between 2 cmH₂O positive end-expiratory pressure when fully open and graduated to 25 cmH₂O fully closed.

8. An anaesthesia non-rebreathing circuit as claimed in claim 1, wherein said smaller-sized mammal has a mass of between 0.5 and 5 kg.

9. An anaesthesia non-rebreathing circuit as claimed in claim 2, wherein said patient adaptor is a connector piece that connects said first conduit and said second conduit to an endotracheal tube for use with said smaller-sized mammal, said connector piece having a gallery from which radiate first, second and third tube connectors, said first tube connector disposed between said second and third tube connectors, said first tube connector having a first longitudinal axis and being adapted to connect to said first conduit, said second tube connector having a second longitudinal axis and being adapted to connect to said second conduit, and said third tube connector having a third longitudinal axis and adapted to connect to said endotracheal tube, said third tube connector being disposed at an obtuse angle relative to said first tube connector with said third longitudinal axis intersecting said first longitudinal axis within said gallery, and said second tube connector disposed at an acute angle relative to said first tube connector with said second longitudinal axis intersecting said first longitudinal axis outside of said connector piece.

10. An anaesthesia non-rebreathing circuit as claimed in claim 9, wherein said third tube connector of said connector piece being disposed at an angle of about one hundred and ten degrees relative to said first tube connector and said second tube connector disposed at an angle of about thirty-five degrees relative to said first tube connector.

11. An anaesthesia non-rebreathing circuit as claimed in claim 9, wherein said third tube connector being disposed at an angle of about one hundred and forty-five degrees relative to said second tube connector.

12. An anaesthesia non-rebreathing circuit as claimed in any of claim 9, wherein said endotracheal tube which connects to said third tube connector has an internal diameter of about 5 mm.

13. An anaesthesia non-rebreathing circuit for a smaller-sized mammal having a mass between 0.5 kg and 5 kg, said anaesthesia non-rebreathing circuit is a parallel Lack circuit having an adjustable pressure limited valve disposed at the waste gas port located at the expiratory end of the circuit, with fresh gas delivered by a first conduit from a vaporiser to a patient adaptor, said first conduit is one of two substantially parallel breathing tubes, and the other of the two breathing tubes is a second conduit disposed between said patient adaptor and the expiratory end of the circuit, wherein said circuit having a heating arrangement for heating gas within said first conduit, said heating arrangement comprises a heating element disposed on said first conduit, operably connected to a thermostatic control unit.

14. An anaesthesia non-rebreathing circuit as claimed in claim 13, wherein said heating element is a spiral wound element extending along a substantial portion of said first conduit.

15. An anaesthesia non-rebreathing circuit as claimed in claim 13, wherein said heating element is a strip element extending along a substantial portion of said first conduit.

16. An anaesthesia non-rebreathing circuit as claimed in claim 13, wherein said patient adaptor is a connector piece that connects said first conduit and said second conduit to an endotracheal tube for use with said smaller-sized mammal, said connector piece having a gallery from which radiate first, second and third tube connectors, said first tube connector having a first longitudinal axis and being adapted to connect said first conduit, said second tube connector having a second longitudinal axis and being adapted to connect to said second conduit, and said third tube connector having a third longitudinal axis and adapted to connect to said endotracheal tube, said gallery and said first and third tube connectors in combination provide an elbow-like change in direction within said connector piece, with said third longitudinal axis intersecting said first longitudinal axis within said gallery, and said second longitudinal axis intersecting said first longitudinal axis outside of said connector.

17. An anaesthesia non-rebreathing circuit as claimed in claim 16, wherein said elbow-like change in direction allows for orientation of breathing tubes relative to said endotracheal tube without the need of an elbow external of said connector piece, thus minimising mechanical dead space between said endotracheal tube and said breathing tubes.

18. An anaesthesia non-rebreathing circuit as claimed in claim 16, wherein said third tube connector being disposed at an obtuse angle relative to said first tube connector with said third longitudinal axis intersecting said first longitudinal axis within said gallery and said second tube connector disposed at an acute angle relative to said first tube connector with said second longitudinal axis intersecting said first longitudinal axis outside of said connector.

19. An anaesthesia non-rebreathing circuit as claimed in any of claim 16, wherein said third tube connector being disposed at an angle of about one hundred and ten degrees relative to said first tube connector and said second tube connector disposed at an angle of about thirty-five degrees relative to said first tube connector.

20. An anaesthesia non-rebreathing circuit as claimed in claim 16, wherein said third tube connector being disposed at an angle of about one hundred and forty-five degrees relative to said second tube connector.
